(19) United States
(12) Patent Application Publication
(10) Pub. No.: US 2023/0355443 A1
(43) Pub. Date: Nov. 9, 2023

(54) SYSTEMS AND METHODS FOR DELIVERING AN ANESTHETIZING SOLUTION INTO THE EAR CANAL

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Rohit K. GIROTRA, San Francisco, CA (US); Eric A. GOLDFARB, Belmot, CA (US); Brian J. DOMECUS, Menlo Park, CA (US); Matthew D. MERTZ, LOS ALTOS, CA (US); Mei PADER, LOS ALTOS, CA (US); Andrew G. LANTZ, Redwood City, CA (US)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 18/245,119

(22) PCT Filed: Sep. 2, 2021
(86) PCT No.: PCT/US21/48798

§ 371 Date: Mar. 13, 2023

Related U.S. Application Data
(60) Provisional application No. 63/084,804, filed on Sep. 29, 2020.

(51) Int. Cl. A61F 11/00; A61N 1/30; A61N 1/04; A61N 1/05

(52) U.S. Cl. CPC A61F 11/00; A61N 1/306; A61N 1/0448; A61N 1/0526; A61F 2250/0067

(57) ABSTRACT

Delivering an anesthetizing solution into the ear canal. One example embodiment is a method of treatment, the method comprising: adhering a pad to a patient, the adhering on the patients face proximate to a tragus of the patient, and the pad being a member of an earset assembly; inserting an earset into an ear canal of the patient; locking relative orientations of the earset and pad by way of a connection assembly, the locking in place to hold the earset in the ear canal; filling the ear canal with therapeutic fluid; and performing iontophoresis with the therapeutic fluid by way of an electrode of the earset.

### CROSS-REFERENCE TO RELATED APPLICATIONS

This application claims the benefit of U.S. Provisional App. No. 63/084,804 filed Sep. 29, 2020 titled “Systems And Methods For Delivering An Anesthetizing Solution Into The Ear Canal” The provisional application is incorporated by reference herein as if reproduced in full below.

### BACKGROUND

Otitis media (“ear infection”) is among the most common diagnoses made by pediatricians. A majority of children have at least one episode of otitis media prior to their third birthday. Otitis media is often caused by an inability of the Eustachian tube to drain fluid from the middle ear, and in many cases is treated with antibiotics.

A significant number of children exhibit recurrent episodes of otitis media and/or otitis media with effusion. Treatment of these more severe cases often involves the placement of a tympanostomy tube across the tympanic membrane to provide adequate drainage and/or ventilation of the middle ear and reduce the likelihood of future infections. Tympanostomy tubes provide fluid communication between the middle and outer ear (e.g., pressure equalization) and in most cases fall out spontaneously within about a year of placement. Tympanostomy tube placement is among the most frequent surgical procedures performed in the pediatric population. It has been estimated that more than a million tympanostomy tubes may be placed each year, with typical patients being between about 18 months and 7 years of age at the time of the procedure.

Systems and methods have been proposed for deploying tympanostomy tubes without having to use general anesthesia, and instead using anesthetizing solutions in the ear canal. It would be desirable to provide improved devices, systems, and methods for delivering an anesthetizing solution into the ear canal.

### SUMMARY

On example embodiment is an earset assembly comprising: a pad defining an adhesive side having an adhesive, a non-adhesive side opposite the adhesive side; an arm defining a proximal end and a distal end, the proximal end coupled to the non-adhesive side of the pad, and an earset. The earset may comprise: a rigid tube defining a proximal end, a distal end, and an internal volume; a nozzle protruding from the internal volume and through the distal end of the rigid tube; an electrode disposed within the internal volume; and the proximal end of the rigid tube coupled to the arm.

The example earset assembly may further comprise a pad connector coupled between the proximal end of the arm and the non-adhesive side of the pad, the pad connector defining an axis of rotation for the arm, and the axis of rotation intersects the adhesive side of the pad.

The example earset assembly may further comprise a pad connector coupled between the proximal end of the arm and the non-adhesive side of the pad, the arm has a single degree of freedom relative to the pad.

The example earset assembly may further comprise an arm connector coupled between the arm and the earset, the arm connector provides the earset only three degrees of freedom relative to the arm.

The example earset assembly may further comprise an arm connector coupled between the arm and the earset, the arm connector forming a ball and a socket arrangement. The socket may protrudes from a distal surface of the arm, and the ball protrudes may from the proximal end of the earset. In some cases, the ball is separable from the socket without damaging the ball or socket. The example earset may further comprise a sound damper disposed between the ball and the socket arrangement. The sound damper may be at least one selected from a group comprising: disposed on an outside surface the ball; and disposed on an inside surface of the socket.

The example earset assembly may further comprise: a slot defined through the arm; an arm connector comprising a connector member abutting a patient side of the arm, and a knob member abutting the clinician side of the arm. The arm connector may comprise a first configuration in which the arm connector slides along the slot, and a second configuration in which the arm connector is rigidly coupled to the slot. A length of the slot may run parallel to a length of the arm, and wherein the slot may intersect the distal end of the arm.

The earset of the example earset assembly may further comprise an ear plug concentrically arranged over a distal end of the rigid tube. The ear plug may comprise: a tube portion defining an internal lumen and a longitudinal central axis, an inside diameter of the tube portion coupled to an outside diameter of the rigid tube of the earset; a seal portion coupled to a distal end of the tube portion, and the seal portion extending proximally over the tube portion; and the tube portion comprising a means for radial reinforcement to resist deformation in radial directions relative the longitudinal central axis. The means for radial reinforcement may comprise a plurality of spokes coupled between an inside surface of the seal portion and an outside surface of the tube portion. In other cases, the means for radial reinforcement may comprise the tube portion having a durometer rating higher than a durometer rating of the seal portion. In some cases, the durometer rating of the tube portion is at least twice the durometer rating of the seal portion. In a particular case, the durometer rating of the tube portion is about 60 Shore A. In some cases, the durometer rating of the seal portion is between and including 10 and 30 Shore A.

The earset of the example earset assembly may further comprise: a vent tube defining an internal vent path, a first end of the internal vent path fluidly coupled to the internal volume of the rigid tube; and a check valve coupled to a second end of the internal vent path, the check valve configured to allow fluid to pass only out of the internal vent path on the second end. The earset may further comprise a reservoir disposed between the internal vent path and the check valve. In some cases, the reservoir is at least partially transparent. In other cases, the earset may further comprise a reservoir disposed downstream of the check valve, and the reservoir may be at least partially transparent.

The earset of the earset assembly may further comprise a fill tube defining an internal flow path, a first end of the fill tube coupled to the internal volume of the rigid tube, and a second end coupled to a fill connector. In some cases, the vent tube may be at least five times longer than a length of the rigid tube of the earset.

The pad of the example earset assembly may further comprise a width and a length, and wherein the length is at least three times the width.

The pad of the example earset assembly may further comprise: a first pad member, the first pad member having a first portion of the adhesive; a second pad member distinct from the first pad member, the second pad member having a second portion of the adhesive; a connection member defining a radius of curvature, the connection member having a first end coupled to the first pad member, and the connection member having a second end coupled to the second pad member, the second end opposite the first end.

Another example embodiment is a method of treatment, comprising: adhering a pad to a patient, the adhering on the patient's face proximate to a tragus of the patient, and the pad being a member of an earset assembly; inserting an earset into an ear canal of the patient; locking relative orientations of the earset and the pad by way of a connection assembly, the locking in place to hold the earset in the ear canal; filling the ear canal with therapeutic fluid; and performing iontophoresis with the therapeutic fluid by way of an electrode of the earset.

Adhering in the example method may further comprise removing a protective covering from adhesive on a patient side of the pad, and pressing the patient side of the pad against the patient's face proximate to the tragus.

In the example method: wherein adhering the pad may further comprise adhering the pad with an arm coupled to the pad and with the earset de-coupled from the arm; and locking relative orientations may further comprise adjusting location of the arm and coupling the earset to the arm. Adjusting location of the arm may further comprise rotating the arm about an axis of rotation, the axis of rotation extends through the pad. Locking relative orientations may further comprise coupling the earset to the arm. Coupling the earset to the arm may further comprise coupling a ball and socket arrangement. In some cases, the ball may disposed on a proximal end of the earset, and the socket may be coupled to the arm.

Locking relative orientations in the example method may further comprise adjusting location of the connection assembly along an arm, the arm coupled between the earset and the pad. Adjusting location of the connection assembly may further comprise sliding the connection assembly along the arm, and then locking the connection assembly relative the arm.

Adhering the pad and inserting the earset in the example method may further comprise inserting the earset into the ear canal and adhering the pad, with the earset coupled to the pad by way of an arm.

Adhering the pad and inserting the earset in the example method may further comprise adhering the pad and then inserting the earset into the ear canal, and the method then may further comprise coupling the earset to the pad by way of an arm.

The example method may further comprise ceasing the filling after a fluid from the ear canal exits a distal end of a vent tube, the distal end of the vent tube disposed outside an area of the ear of the patient. Ceasing the filling may further comprise ceasing after the fluid enters a reservoir fluidly coupled to the distal end of the vent tube. Ceasing the filling may further comprise ceasing the filling after the fluid passes through a check valve fluidly coupled on the distal end of the vent tube.

### BRIEF DESCRIPTION OF THE DRAWINGS

For a detailed description of example embodiments, reference will now be made to the accompanying drawings in which:

FIG. 1A shows a perspective view of an earset assembly in accordance with at least some embodiments;

FIG. 1B shows a perspective view of an earset assembly in accordance with at least some embodiments;

FIG. 2 shows a perspective view of a pad of an earset assembly in accordance with at least some embodiments;

FIG. 3 shows a perspective view of a portion of an earset in accordance with at least some embodiments;

FIG. 4 shows cross-sectional perspective view of an arm and a pad in accordance with at least some embodiments;

FIG. 5 shows a side view of an arm and socket in accordance with at least some embodiments;

FIG. 6 shows an exploded side elevation view of an earset in accordance with at least some embodiments;

FIG. 7 shows a cross-section perspective view of an earset in accordance with at least some embodiments;

FIG. 8 shows a cross-sectional view of an ear plug in accordance with at least some embodiments;

FIG. 9 shows a cross-sectional perspective view of a vent assembly in accordance with at least some embodiments.

FIG. 10 shows a cross-sectional perspective view of a vent assembly in accordance with at least some embodiments;

FIG. 11 shows a perspective view of a pad system in accordance with at least some embodiments;

FIG. 12 show a back perspective view of an ear plug in accordance with at least some embodiments;

FIG. 13 shows a back perspective view of an ear plug in accordance with at least some embodiments;

FIG. 14 shows a cross-sectional side view of an ear plug in accordance with at least some embodiments; and

FIG. 15 shows a method in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“About” in reference to a recited value shall mean the recited value plus or minus 5% of the recited value.

Various apertures may be referred to as a “bore,” “through bore”, or “counter bore.” Reference to a bore, through bore, or counter bore shall not be read to imply that any such bore, through bore, or counter bore is created by boring or drilling. The bore, through bore, or counter bore may be created in any suitable fashion, including not only drilling and boring, but also milling, casting, and laser cutting, to name a few.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various example embodiments are directed to systems and methods for delivering an anesthetizing solution into the ear canal. In particular, various example embodiments are directed to an earset assembly, and method of using an earset assembly, in which the earset is held in place within the ear canal by a pad (or set of pads) adhered to the skin of the patient's face. Moreover, in the example embodiments venting of excess anesthetizing fluid from the ear canal, such as during filing or when the volume changes based on movement of the patient's jaw, takes place away from the ear such that the excess anesthetizing fluid does not flow onto the ear itself. The description first turns to an example earset assembly.

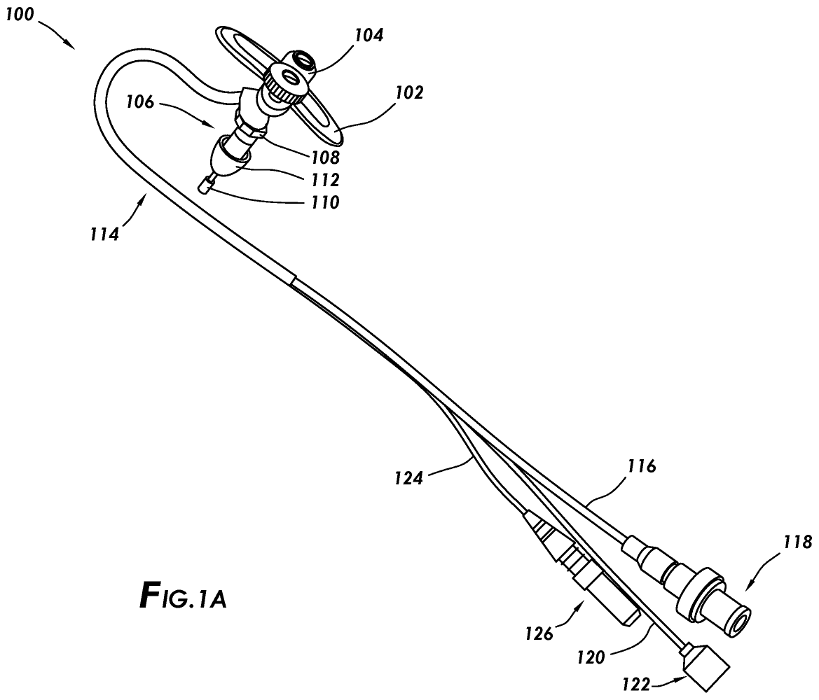

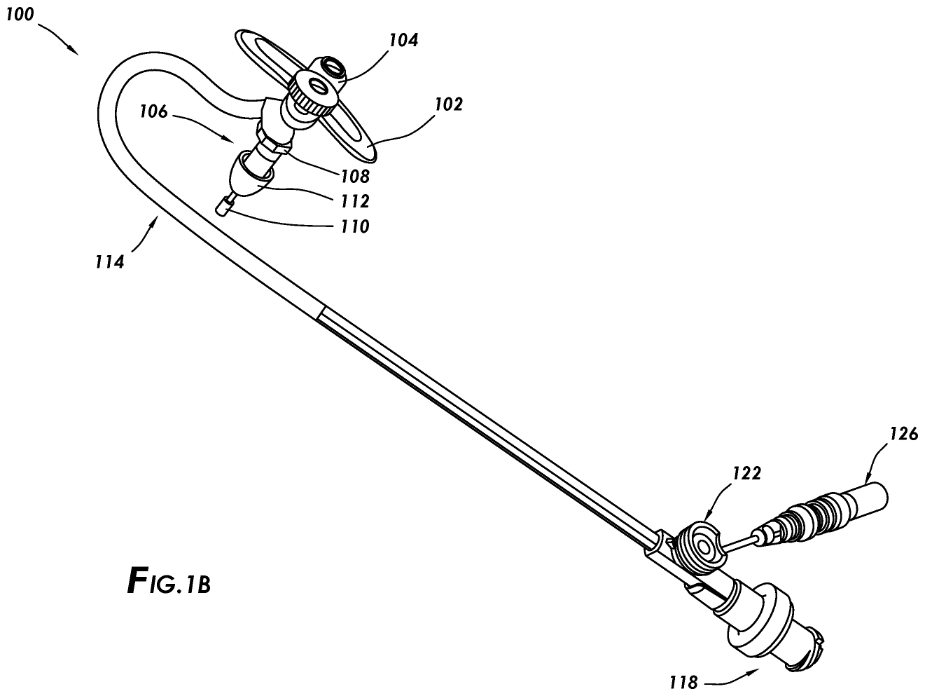

FIG. 1A shows a perspective view of an earset assembly 100 in accordance with at least some embodiments. In particular, the example earset assembly 100 comprises a pad 102, an arm 104, and an earset 106. The example earset 106 comprises a rigid section, distal region, or rigid tube 108 with a nozzle 110 extending therefrom. An ear plug 112 is telescoped over and is concentrically arranged with the rigid tube 108. The earset 106 further comprises a tube set 114 that coupled to a proximal end of the earset 106, and the various tubes of the tube set 114 that extend a distance away from the earset 106 as shown. The example tube set 114 comprises a fill tube 116 terminating in a fill connector 118, a vent tube 120 terminating in a vent assembly 122, and an electrical lead 124 terminating in an electrical connector 126.

FIG. 1B shows a perspective view of another example earset assembly 100. In particular, the example earset assembly 100 of FIG. 1B has all the noted components of the easrset assembly 100 of FIG. 1A, and in the case of FIG. 1B the distal ends of the various members of the tube set 114 are held together by an extension of the housing that forms the vent assembly 122.

In use as part of anesthetizing a tympanic membrane of a patient, the pad 102 is adhered on the patient's head or face near or proximate to the tragus/Preauricular area. The earset 106 is inserted into an ear canal of the patient, and once in the proper orientation, the relative positions of the earset 106 and the pad 102 are locked in place. Thereafter, the ear canal is filled with therapeutic fluid by way of the fill connector 118 and the fill tube 116. Air within the ear canal and the earset 106 is displaced with therapeutic fluid during filling, and the displaced air exits the sealed volume of the ear canal by way of the vent tube 120 and vent assembly 122. Once the ear canal is filled with therapeutic fluid, an electrical current is provided to an electrode (not visible) within the earset 106 by way of the electrical connector 126 and the electrical lead 124. Thus, iontophoresis is performed with the therapeutic fluid by way of the electrode of the earset 106. Once the tympanic membrane is anesthetized, the earset assembly 100 may be removed from the patient, the fluid drained from the ear canal, and additional procedures may take place with respect to the tympanic membrane, such as a tympanostomy in which a vent tube is placed across the tympanic membrane. The specification now turns to a more detailed description of the various components of the earset assembly 100, starting with the pad 102.

FIG. 2 shows a perspective view of a pad 102 of an earset assembly 100 in accordance with at least some embodiments. In particular, the example pad 102 defines a patient side 202 (not visible) having an adhesive disposed thereon, in some cases a pressure sensitive adhesive. The patient side 202 is so named to acknowledge that, in use, the adhesive of the patient side 202 adheres and thus couples to the skin of the face of the patient. The pad 102 further defines a non-adhesive or clinician side 204 opposite the patient side 202. The clinician side 204 is so named to acknowledge that, in use, the clinician side 204 faces away from the patient and thus likely faces the clinician, particularly during installation. In example embodiments, the pad 102 defines an oval shape or kidney-bean shape, but other shapes are possible, including polygonal shapes (e.g., triangles and rectangles). The example shape enables the pad 102, and in fact the entire earset assembly 100, to be used on either side of the patient's face, eliminating the need to have pads designed specifically for use on only one side of the face.

The example pad 102 defines a length L_{P} and a width W_{P} (e.g., an average width, or a peak width). The length L_{P} is greater than the width W_{P}, and in some cases the length L_{P} is at least three times the width W_{P}. In one example case, the length L_{P} is about 1.75 inches (about 4.44 centimeters (cm)), and the width is about 0.5 inches (1.3 cm). The example dimensions result in an area on the patient side 202 of about 0.875 square inches (about 5.77 cm²), though larger or smaller areas may be used. The width Wp may be selected to reduce covering the sideburn hair, and the length L_{P} and may be selected to increase surface area to provide stability by distributing the adhesive force over a larger skin surface area. The example pad 102 is made from a semi-rigid elastomeric material, such as thermoplastic elastomers (TPE), to enable the pad 102 to conform to the contours of the patient's face. In one example system, the pad 102 has a Shore A hardness of about 60.

Still referring to FIG. 2, the example pad 102 further comprises a pad connector 206 medially disposed on the pad 102, though other placements are possible. In particular, the pad 102 defines a reinforcing ridge 208 that runs along the length L_{P} on the clinician side 204 (e.g., runs parallel to the length L_{P}). The reinforcing ridge 208 not only reinforces the pad 102 to reduce flexing along the length L_{P}, but also provides a connection location for the example pad connector 206. The example pad connector 206 defines an axis of rotation 210, and the axis of rotation 210 intersects the patient side 202 of the pad 102. In one example case, the axis of rotation 210 is perpendicular to the patient side 202 of the pad 102. The example pad connector 206 defines a snap arm 212 and a snap arm 214. The snap arms 212 and 214 are designed and constructed to flex inward toward the axis of rotation 210 during assembly of the arm (discussed more below), and then enable the arm to rotate around the axis of rotation 210. In particular, the snap arms 212 and 214 define an annular groove 216 that is perpendicular to the axis of rotation 210, and the arm 104 swings around the pad 102 in the annular groove 216.

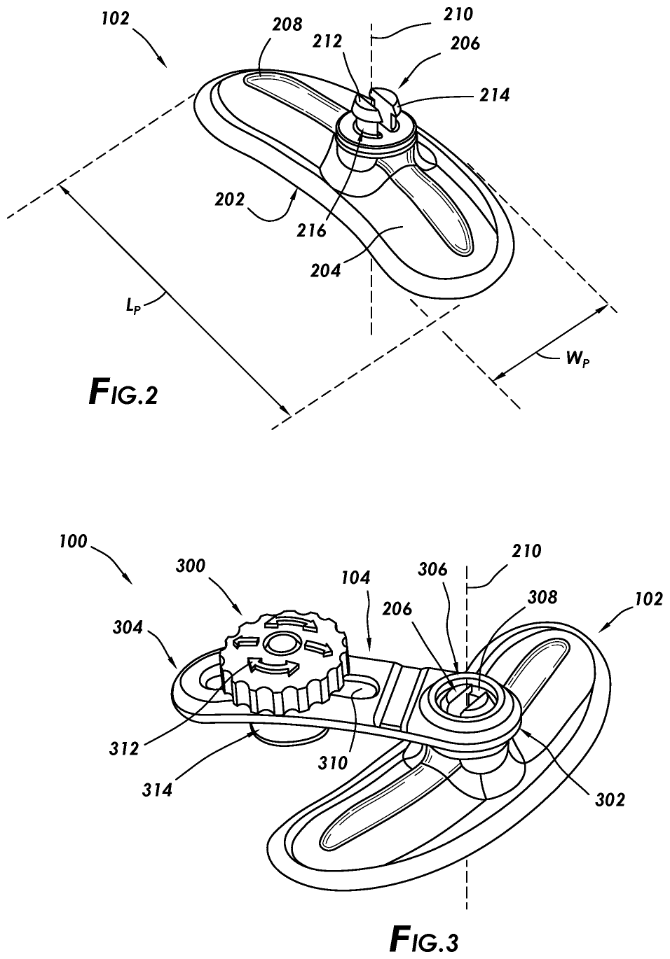

FIG. 3 shows a perspective view of a portion of the earset assembly 100 in accordance with at least some embodiments. In particular, FIG. 3 shows the pad 102 connected to an arm 104, and the arm 104 defines a portion of an arm connector 300. The arm 104 defines a proximal end 302 and a distal end 304. The proximal end 302 defines an aperture 306 that telescopes over the snap arms 212 and 214 of the pad connector 206. In particular, the aperture 306 defines an internal annular ridge 308 that interacts with the annular groove 216 (FIG. 2) to enable the arm 104 to rotate around the axis of rotation 210. Stated differently, the arm 104 has a single degree of freedom with respect to the pad connector 206 and/or the pad 102, the single degree of freedom being rotation around the axis of rotation 210. In other cases, the pad connector 206 may implement more than one degree of freedom for the arm 104, and then once the arm 104 is in place the pad connector 206 locks the relative orientations or positions of the arm 104 and the pad 102.

The example arm 104 defines an aperture or slot 310 through the arm 104. The slot has a long dimension that runs parallel to the length of the arm 104. In the example shown, the slot 310 terminates prior to the distal end 304 of the arm 104. However, in other cases the slot 310 may extend all the way to and intersect the distal end 304 of the arm 104. The portion of the arm connector 300 visible in FIG. 3 includes a release or knob 312 disposed on the clinician side of the arm 104, and socket portion 314 of a ball and socket arrangement extending in the opposite direction (e.g., extending from the patient side of the arm 104). In the example system, a stem of the socket portion 314 extends through the slot 310 and interacts with the knob 312. The knob 312 has a first rotational orientation or first configuration in which the socket portion 314 and knob 312 may slide along the slot 310 for purposes of placement with respect to the earset 106 (FIG. 1). The knob 312 has a second rotational orientation or second configuration in which the socket portion 314 is locked at a particular location along the slot 310. That is, in use the knob 312 is unlocked to enable the socket portion 314 to be placed relative to the arm 104, and as discussed more below the placement over a ball portion of the earset 106. Once in the proper orientation, the knob 312 is turned to lock the location of the socket portion 314 in place along the slot 310 of the arm 104. The ball and socket arrangement, as well as the locking and unlocking implemented by the knob 312, are discussed in greater detail below.

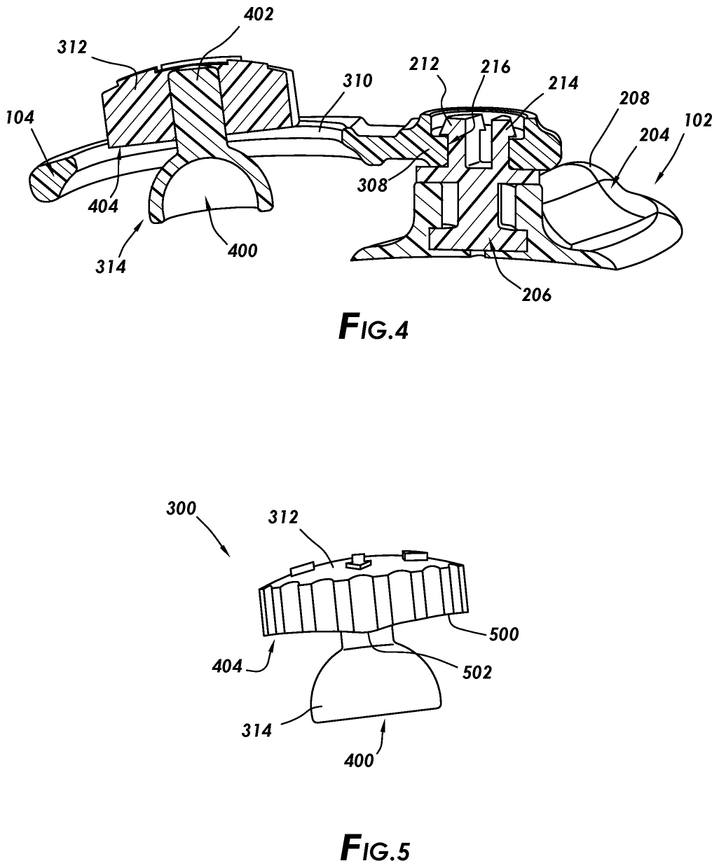

FIG. 4 shows cross-sectional perspective view of the example arm 104 and pad 102. In particular, FIG. 4 shows the pad connector 206 defining the snap arms 212 and 214. The example pad connector 206 of FIG. 4 couples to the pad 102 by having a portion of the pad connector 206 that telescopes into an aperture defined through the clinician side 204 of the pad 102, and within the reinforcing ridge 208. However, any suitable connection system may be used to couple the pad connector 206 to the pad 102, including use of adhesives (e.g., epoxy). The snap arms 212 and 214 define the annular groove 216 into which the internal annular ridge 308 of the arm 104 couples. The interaction of the internal annular ridge 308 and the annular groove 216 enables the single degree of freedom of the arm 104 with respect to the pad connector 206 and/or the pad 102.

The cross-sectional view of FIG. 4 also shows the relationship of the arm connector 300 to the arm 104, including the knob 312 and the socket portion 314. In particular, the socket portion 314 defines a concave or hemispherical socket 400 and a stem 402. The socket 400 is disposed on the patient side of the arm 104, and the socket 400 opens in the direction of the patient side of the arm 104 (e.g., opens toward the patient when present). The stem 402 extends through the slot 310 of the arm 104 and interacts with the knob 312.

While many locking and unlocking mechanisms may be implemented as between the knob 312, the arm 104, the stem 402, and the socket portion 314, one consideration is sound transmission to the ear of the patient. That is, in some situations the adjustment of the location of the knob 312 and socket portion 314 along the arm 104 may take place with the socket 400 coupled to a ball on the earset 106 (FIG. 1). Sharp interactions or snapping noises may be directly conveyed to the patient's ear canal and tympanic membrane along the earset 106, which may startle the patient. Thus, in example systems the configurations of the arm connector 300 that enable movement along the arm 104, and rigidly coupling the arm connector 300 to the arm 104, are implemented in such a way as to reduce or eliminate the noise component when transitioning between the two configurations (e.g., locked and unlocked).

Still referring to FIG. 4, the example arm 104 is curved. That is, the arm 104 defines a radius of curvature, where the center of the radius of curvature is, in the view of FIG. 4, below the patient side of the arm 104. The two example configurations of the arm connector 300 utilize the curvature of the arm. In particular, the knob 312 defines a cam or interaction surface 404 that abuts the clinician side of the arm 104. In one rotational orientation of the knob 312, the interaction surface 404 pinches or squeezes the arm 104 between the outside surface of the socket 400 and the interaction surface 404, rigidly coupling the arm connector 300 in place relative to the arm 104. In another rotational orientation of the knob 312, the interaction surface 404 enables movement of the arm connector 300 along the slot 310.

FIG. 5 shows a side view of the example arm connector 300. In particular, FIG. 5 shows the knob 312 and the socket portion 314. Also visible in the view of FIG. 5 is the profile of the interaction surface 404. The example interaction surface 404 of the knob 312 defines a relatively flat portion 500 and a peak portion 502. In some cases, the knob 312 defines a second peak portion on the opposite side of the knob 312, and thus the second peak portion is not visible in FIG. 5. When knob 312 is turned such that the peak portions reside outside the surface area of the arm 104 on the clinician side of the arm 104, the arm connector 300 may be moved along the slot 310. However, when the knob 312 is turned about a quarter of a turn (e.g., 90 angular degrees), the peak portion 502 and the corresponding peak portion on the opposite side of the knob 312 rid up onto the clinician-side of the arm 104. The alignment pulls the outside surface of the socket 400 against the patient side of the slot 310, which locks the arm connector 300 in place. The peak portion 502 not only provides a relatively smooth transition between the locked and unlocked configurations, but reduces or eliminates snapping noises, and also provides tactile feedback regarding rotational position. The specification now turns to a more detailed description of the example earset 106.

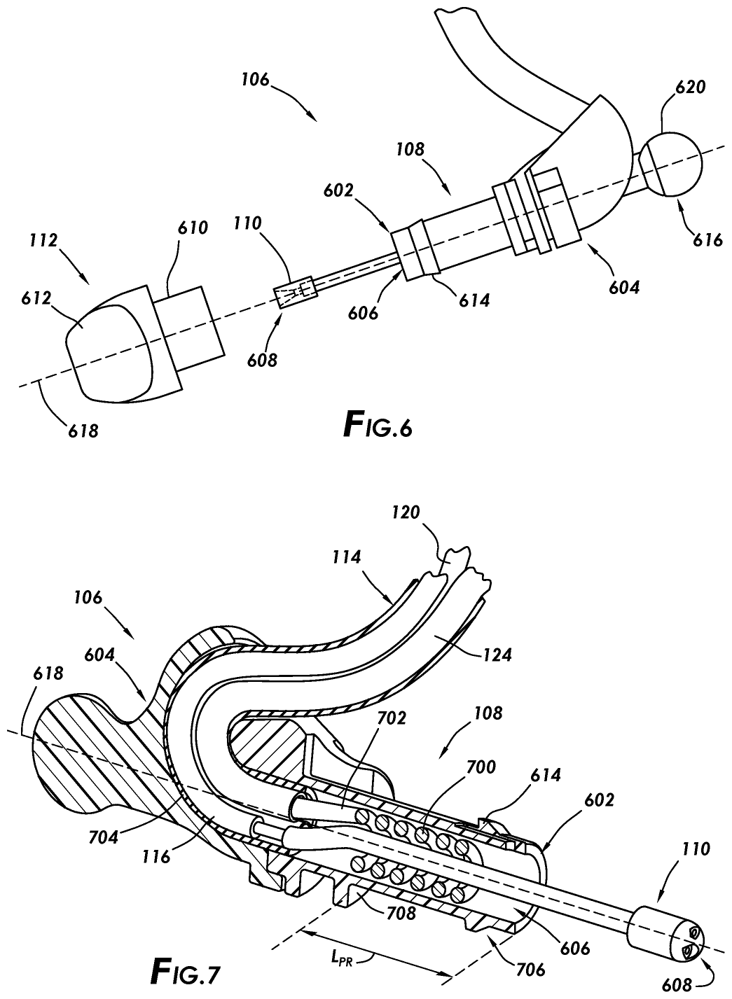

FIG. 6 shows an exploded side-elevation view of the example earset 106. In particular, FIG. 6 shows the rigid tube 108 that defines a distal end 602, a proximal end 604, and an internal volume 606. The nozzle 110 protrudes from the internal volume 606 through the distal end 602 of the rigid tube 108. The nozzle 110 is fluidly coupled to the fill tube 116 (FIG. 1) such that therapeutic fluid injected into the fill connector 118 (FIG. 1) flows through the fill tube 116, into an internal lumen of the nozzle 110, and then exits the nozzle 110 through ports on the distal end 608 of the nozzle 110. In use, the therapeutic fluid fills the ear canal starting approximately at the tympanic membrane by operation of the nozzle 110.

The ear plug 112 defines a tube portion 610 and a seal portion 612. The tube portion 610 defines an internal lumen (not visible in FIG. 6), and when assembled an inside diameter of the tube portion 610 is coupled to an outside diameter of the rigid tube 108. In particular, the tube portion 610 of the ear plug 112 telescopes over and is concentrically arranged with the outside diameter of the rigid tube 108. The example outside diameter of the rigid tube 108 defines an annular ridge 614 that interacts with the insider diameter of the tube portion 610 to help hold the ear plug 112 in place during installation and removal. It follows that the example ear plugs are interchangeable. The example ear plug 112 is discussed in greater detail below.

Still referring to FIG. 6, in the example system the proximal end 604 is coupled to a ball 616, where the ball 616 can be considered a member of the arm connector 300 (FIG. 3). In particular, the ball 616 is disposed on the proximal end 604 of the rigid tube 108, and when the earset 106 is (FIG. 1) is assembled into the earset assembly 100 (FIG. 1) the ball 616 protrudes toward the arm 104. The ball 616 and the corresponding socket 400 are designed and constructed such that when interfaced with the pad and arm the ball and socket apply an inward force (extended from the pad/arm) tending to hold the earset 106 in the ear canal of the patient. To that end, the example ball 616 has a central axis that is coaxial with the longitudinal central axis 618 of the rigid tube 108 and the ear plug 112. While in the example system the ball 616 is rigidly coupled to the earset 106 and the socket 400 is coupled to the arm 104, in other cases the locations may be swapped. Thus, in other cases the socket may be rigidly coupled to the earset 106 and projected or opening toward the arm 104, and the ball may be coupled to the arm 104 and extend away from the patient side of the arm 104.

In the example system, the ball 616 and the socket 400 (FIG. 4) are designed and constructed to be separable. Thus, during installation of the earset 106 into the ear canal of a patient, the ball 616 and the socket 400 may be separated, and the arm 104 (FIG. 1) rotated out of the path of insertion of the earset 106 into the ear canal. Once the earset 106 is in place in the ear canal, the arm 104 may be swung back into position such that the socket 400 is placed over the ball 616, and the socket 400 is locked in place by turning the knob thus locking the earset 106 in place in the ear canal. In other cases, however, the ball and socket arrangement may be pre-assembled and non-separable. In the non-separable case, the earset 106 may be telescoped within the ear canal of the patient with the arm 104 and pad 102 connected to the earset 106. Thus, installation of the earset 106 in the ear canal and adhesion of the pad 102 to the face of the patient may take place substantially simultaneously.

Still referring to FIG. 6, and considering the embodiments in which the ball and socket arrangement is separable by the clinician, coupling the socket 400 (FIG. 4) to the ball 616 has the potential to make a clicking or snapping noise. As previously discussed, noises created by the earset assembly 100 may be directly conveyed to the patient's ear canal and tympanic membrane along the earset 106, which may startle the patient. Thus, in example systems the socket is a slip-fit over the ball—the features to not snap together to reduce noise generation. Moreover, in some example cases a sound damper is disposed between the ball and socket arrangement. In the example of FIG. 6, the sound damper 620 is disposed on at least a portion of an outside surface of the ball 616. The sound damper 620 may take any suitable form, such as an elastomeric material (e.g., TPE) installed in any suitable form, for example, over-molded over the ball 616. The sound damper 620 thus reduces or eliminates sound created when the socket 400 is placed over the ball. Alternatively or additionally, the sound damper may be disposed within the concave region (e.g., the inside surface) of the socket 400. The specification now turns to a more detailed description of an example internal arrangement of the earset 106.

FIG. 7 shows a cross-section perspective view of an example earset 106, without the ear plug installed. In particular, FIG. 7 shows the rigid tube 108 defining the internal volume 606. The nozzle 110 extends from within or protrudes from the internal volume 606 through the distal end 602 of the rigid tube 108. The rigid tube 108 defines a plug region 706. In particular, the plug region 706 extends from a medial shoulder 708 to the distal end 602 of the rigid tube 108, and the plug region 706 includes the annular ridge 614. Thus, the plug region 706 define length L_{PR} measured parallel to the longitudinal central axis 618. As will be discussed in greater detail below, the ear plug 112 telescopes over the length L_{PR} and extends beyond or has an overhang in the distal direction (e.g., toward the distal end of the nozzle 110).

The internal lumen of the nozzle 110 is fluidly coupled to the fill tube 116 such that, during filling of the ear canal the therapeutic fluid is delivered through the fill tube 116 and nozzle 110 toward the tympanic membrane. Air displaced by the therapeutic fluid during filling exits the sealed volume of the ear canal by way of the internal volume 606 and the vent tube 120. That is, displaced air enters the internal volume 606, and then enters to the aperture associated with the vent tube 120.

Once the ear canal and the internal volume 606 of the earset 106 are filled with therapeutic fluid, iontophoresis takes place by application of voltage and corresponding current applied to the therapeutic fluid. The electrical current drives ions of the therapeutic fluid (e.g., iontopheretic solution) into the tympanic membrane. In the example system, the voltage and current is provided to an electrode 700 disposed within the internal volume 606. The example electrode 700 is electrically coupled to an electrical conductor 702 of the electrical lead 124. Thus, by virtue of the electrical conductor 702 and electrode 700, voltage can be applied to the therapeutic fluid in the ear canal and a corresponding current can be driven to perform the iontophoresis.

In some example systems, and as shown in FIG. 7, the electrode 700 is implemented in the form of coil of metallic material, where the coil of metallic material circumscribes the lumen of the nozzle 110. Stated otherwise, the coil has a longitudinal central axis that is coaxial with the longitudinal central axis 618 of the rigid tube 106. However, other arrangements are possible, including arrangements in which the longitudinal central axis of the coil of metallic material is parallel to but not coaxial with the longitudinal central axis 618. In some cases the coil of metallic material is coil of silver to reduce electrolysis; however, other metallic substances, including alloys, may be used. Moreover, while FIG. 7 shows the electrode 700 as a coil, in yet still other cases the electrode 700 may be implemented as a sheet or tube of metallic material within the internal volume 606 and electrically coupled to the electrical conductor 702 associated with the electrical lead 124.

Still referring to FIG. 7, the example earset 106 is coupled to the tube set 114. In particular, the tube set 114 enters the proximal end 604 of the earset 106 by way of channel or lumen 704 defined within the proximal end 604. The tube set 114 comprises the fill tube 116, the vent tube 120, and the electrical lead 124, and possibly an outer cover of non-conductive material (e.g., heat shrink tubing). The fill tube 116 and the vent tube 120 terminate within the internal volume 606. The fill tube 116 is fluidly coupled to the lumen of the nozzle 110. The vent tube 120 is fluidly coupled to the internal volume 606 of the rigid tube 108. The electrical lead 124 includes an insulating sheath, and the insulating sheath terminates at any suitable location that enables the electrical conductor 702 to electrically couple to the electrode 700. The example lumen 704 defines curved path to enable the tube set 114 to be routed over the pinna. However, the curvature of the lumen 704 is not strictly required, and thus any suitable arrangement that enables the tube set 114 to couple to the earset 106, yet reduce interaction with the arm 104 (FIG. 1) and arm connector 300 (FIG. 3), may be used.

During filling of the ear canal, one parameter of interest is the increase in pressure of the fluid in the ear canal over atmospheric pressure. Stated otherwise, one consideration is the amount of differential pressure applied across the tympanic membrane caused by the fill procedure. In accordance with example systems, in order to limit the differential pressure applied across the tympanic membrane during filling, the cross-sectional area of the flow path of fluid into the ear canal (e.g., the inside diameter of the lumen of the nozzle 110, and/or combined cross-sectional area of the ports through the nozzle 110) should be the same or smaller than the cross-sectional area of the vent path out of the earset 106. Stated otherwise, by limiting the cross-sectional fill area to be the same or smaller than the cross-sectional vent area, the rate of change pressure across the tympanic membrane can be limited. In one example case, the inside diameter of the fill tube 116 is between and including 0.0028 inch (0.071 millimeter (mm)) and 0.0038 inch (0.097 mm), and in one case the inside diameter is about 0.0031 inch (about 0.078 mm). Correspondingly, the inside diameter of the vent tube 120 is the same or larger. The length of the vent tube 120 may also be a consideration. That is, a vent tube 120 that is significantly longer than the fill tube 116 may result in excess back pressure to vent flow of therapeutic fluid. However, in example embodiments the length issues is obviated by have the vent tube 120 either be shorter than the fill tube 116, or the having the vent tube 120 and fill tube 116 being approximately the same length. The specification now turns to a discussion of ear plugs in accordance with example embodiments.

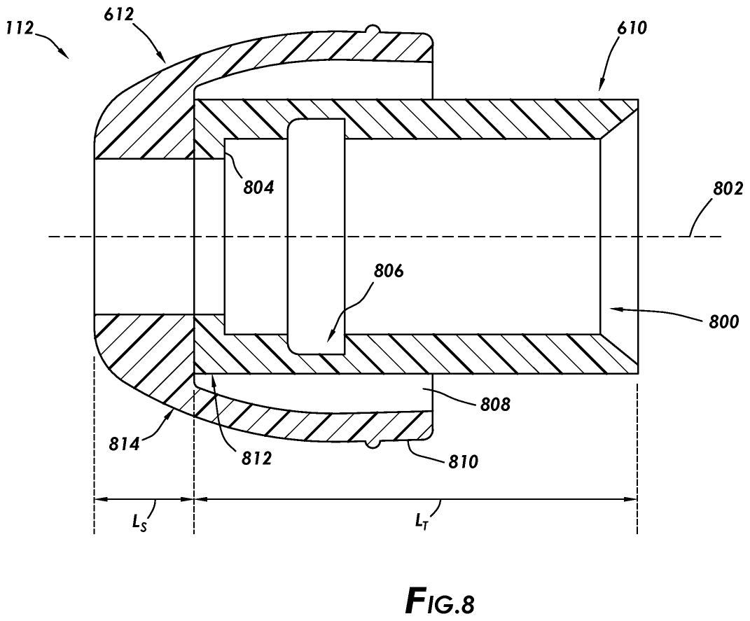

FIG. 8 shows a cross-sectional view of an example ear plug 112. In particular, FIG. 8 shows the tube portion 610 and the seal portion 612. The tube portion 610 defines an internal lumen 800 and a longitudinal central axis 802. When the ear plug 112 is coupled to the rigid tube 108 (FIG. 7) of the earset 106, an inside diameter of the tube portion 610 is coupled to and abuts an outside diameter of the rigid tube 108. Moreover, in example cases, when the ear plug 112 is coupled to the rigid tube 108, the longitudinal central axis 802 is coaxial with the longitudinal central axis 618 (FIG. 6) of the rigid tube 108. The inside diameter of the example tube portion 610 includes a counter bore that defines an annular shoulder 804 near the distal end of the internal lumen 800. Also defined on the inside diameter of the internal lumen 800 is an annular channel 806, and the annular channel 806 is disposed proximally from the annular shoulder 804.

The example ear plug 112 further comprises the seal portion 612. The seal portion 612 is coupled to the distal end of the tube portion 610. The example seal portion 612 extends distally from the tube portion 610, but also extends proximally over the tube portion 610. Thus, the seal portion 612 defines an “umbrella” shape including an inside surface 808 that faces the outside diameter or outside surface of the tube portion 610. Stated otherwise, the seal portion 612 defines a frusto-conical outside surface 810, and a corresponding frusto-conical inside surface.

The tube portion 610 has a length L_{T} measured parallel to the longitudinal central axis 802. In example cases, the length L_{T} is longer than the length L_{PR} (FIG. 7) of the rigid tube 108 such that the tube portion 610 defines an extension or overhang 812 beyond the distal end of the rigid tube 108 (e.g., that portion distal to the annular shoulder 804). Moreover, the seal portion 612 extends distally beyond the end of the tube portion 610 and thus also defines an extension or overhang 814, and the overhang 814 defines a length L_{S} measured parallel to the longitudinal central axis 802. The overhangs 812 and 814 of the tube portion 610 and seal portion 612, respectively, form a protective elastomeric shield over the distal end of the rigid tube 108 to ensure the distal end of the rigid tube does not contact the patient during installation.

In example embodiments, the ear plug 112 used with each earset 106 includes radial reinforcement to resist collapse or deformation toward the longitudinal central axis 802 of the ear plug 112. More particularly, in example cases the ear plug 112 has radial reinforcement that performs several functions. The radial reinforcement is designed and constructed to resist deformation of the tube portion 610 during installation of the ear plug 112 on the rigid tube 108. Resisting deformation during installation of the ear plug 112 on the rigid tube 108 reduces or eliminates the possibility of pushing the distal end of the rigid tube 108 beyond the annular shoulder 804 within the tube portion 610. Stated differently, the radial reinforcement helps provide a tactile feedback to the clinician installing the ear plug 112 to ensure the tube portion 610 and rigid tube 108 are properly aligned axially. When the distal end of the rigid tube 108 abuts the shoulder 804 and/or the annular ridge 614 (FIG. 6) of the rigid tube 108 slides into the annular channel 806 of the tube portion 610, sliding into those mechanical relationships provides tactile feedback to the clinician. Relatedly, the radial reinforcement reduces or eliminates the chances of the ear plug 112 slipping off the rigid tube 108 during removal of the earset 106 from the ear canal of the patient.

Additionally, the radial reinforcement may resist deformation of the overhang 812 of the tube portion 610 when the earset 106 is placed within the ear canal of the patient. Resisting deformation of the overhangs 812 and 814 may also help hold the overhangs 812 and 814 in a more circular configuration, and thus ensures the lumen defined by the overhangs 812 and 814 remain open to flow of displaced air and therapeutic fluid. Moreover, the radial reinforcement may reduce an amount of collapse or compression experienced by the seal portion 612 when the seal portion 612 is pushed toward the tube portion 610 during installation into the ear canal of the patient. For example, the radial reinforcement may reduce the movement of the inside surface 808 of the seal portion toward the outside diameter of the tube portion 610, and/or may reduce the amount of collapse or compression experienced by the either or both the seal portion 612 and tube portion 610 if the inside surface 808 contacts the outside diameter of the tube portion 610.

In some example embodiments, the radial reinforcement is implemented in the form a dual-durometer ear plug. Still referring to FIG. 8, the example ear plug 112 implements the radial reinforcement by implementing the tube portion 610 and the seal portion 612 in elastomeric material having different hardness. In one example, the tube portion 610 has a durometer rating higher than a durometer rating of the seal portion 612. In some cases, the durometer rating of the tube portion 610 is at least twice the durometer rating of the seal portion 612. In a specific case, the tube portion 610 has a hardness of about 60 Shore A, and the seal portion 612 has a hardness of between and including 10 and 30 Shore A. Other arrangements are possible and are discussed more below.

Returning briefly to FIG. 1. The example earset assembly 100 includes the tube set 114 coupled to the earset 106. The tube set 114 includes the vent tube 120 and vent assembly 122. The vent tube 120 is mechanically and fluidly coupled to the vent assembly 122. The specification now turns to a more detailed discussion of example vent assembly 122.

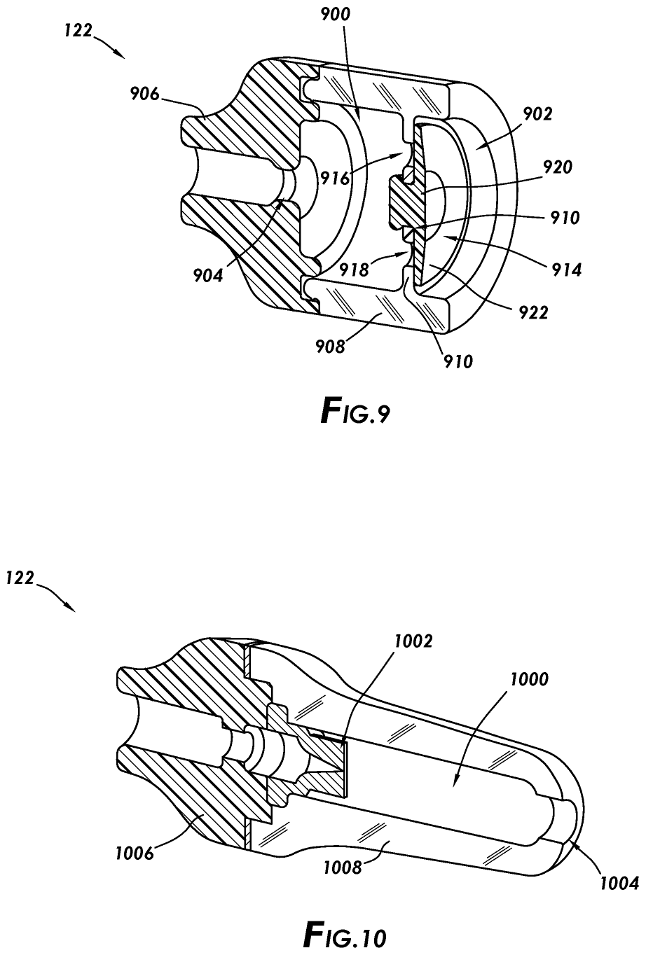

FIG. 9 shows a cross-sectional perspective view of the example vent assembly 122. In particular, the example vent assembly 122 comprises a reservoir 900 and a check valve 902. The example reservoir 900 is fluidly coupled to the vent tube 120 (FIG. 1) such that, during filling of the ear canal with therapeutic fluid, displaced air and excess therapeutic fluid flow through the vent tube 120 and then enter the reservoir 900. In particular, displaced air and excess therapeutic fluid enter the reservoir 900 by way of the aperture or inlet 904. As the therapeutic fluid flows into the ear canal and then into the vent tube 120, the displaced air is vented through the check valve 902 to atmosphere. However, as the name implies, the check valve 902 does not allow reverse flow of air or liquids. Further, the excess therapeutic fluid flows into the vent tube 120 and enters the reservoir 900, and if therapeutic fluid continues to be provided to the ear canal, the excess therapeutic fluid is also vented through the check valve 902.

In accordance with example embodiments, the reservoir 900 is implemented in a transparent material such that the clinician, injecting the therapeutic fluid into the ear canal (e.g., by way of the fill connector 118 and fill tube 116) can monitor the reservoir 900. When therapeutic fluid arrives in the reservoir 900, the clinician may assume the ear canal has been fully filled with the therapeutic fluid, and thus filling may stop and the iontophoresis may proceed. In some cases, the filling of the ear canal can thus occur without venting of therapeutic fluid through the check valve 902. Even if some venting of therapeutic fluid through the check valve 902 occurs, such is a minor concern since the venting is away from the patient's ear and the vented fluid cannot be confused with leakage from the ear canal. That is, in some cases the vent tube 120 is at least five times longer than the length of the rigid tube 108 of the earset 106 such that any venting of fluid occurs well away from the location of the patient's ear.

The example vent assembly 122 defines a coupler or connector 906. The connector 906 is designed and constructed to mechanically and fluidly couple to the vent tube 120 such that displaced air and excess therapeutic fluid may flow through the inlet 904 into the reservoir 900. In some cases, the connector 906 is constructed of transparent material to help enable visualization into the reservoir 900, but in other cases the connector is opaque and visualization is implemented by way of other components. The example vent assembly 122 further comprises a cylinder or tube member 908. The tube member 908 couples to the connector 906 on a first end, and in the example case the tube member 908 defines the stationary portion of the check valve 902 on the second end, opposite the first end. The example tube member 908 is constructed of transparent material to enable visualization into the reservoir 900 by the clinician. The tube member 908 defines a wall member 910 opposite the inlet 904, and thus the reservoir is defined in part by the connector 906, the inside diameter of the tube member 908, and the inside surface of the wall member 910.

The wall member 910 further includes a plurality of apertures. In particular, an example connector aperture 912 is centered in the wall member 910 and defines the location that the flap member 914 of the check valve 902 couples and is held in place with respect to the wall member 910. The example wall member 910 further defines a plurality of vent apertures. In the view of FIG. 9, two partial vent apertures are shown, being vent aperture 916 and vent aperture 918. One or more vent apertures may be used, and in some case six vent apertures are implemented.

The example flap member 914 has a center post or connector 920 that telescopes through the connector aperture 910 to hold the flap member 914 in place. Surrounding the connector 920 is a valve member, flap, or annular seal 922 that defines a diameter large enough to cover the vent apertures (e.g., vent apertures 916 and 916). When displaced air and excess therapeutic fluid needs to escape the reservoir 900, the air/fluid flows through one or more of the vent apertures, pushing open the annular seal 922. However, the flap member 914, and particularly the annular seal 922, seals against the vent apertures to prevent flow of air or liquids back into the reservoir 900. In some cases, and as shown in FIG. 9, the flap member 914 is recessed to reduce or prevent user interaction with the valve and/or occlusion.

The reservoir 900 may have any suitable volume. In an example, the volume of the reservoir 900 may be between and including 0.100 cubic centimeters (cc) and 0.300 cc. Correspondingly, the flap member 914 may take any suitable size to operate as a check valve with respect to the flow of air and fluid through apertures in the wall member 910. In one example case, the flap member 914 may have a diameter of about 0.236 inches (about 0.600 cm); however, the diameter of the flap member 914 is related to dimensions of the tube member 908 that defines the reservoir 900.

In the example vent assembly 122 of FIG. 9, the reservoir 900 is fluidly upstream from the check valve 902. While the arrangement of having the reservoir 900 provides certain advantages (e.g., the excess fluid can be seen by the clinician before fluid passes the check valve), other arrangements are possible.

FIG. 10 shows a cross-sectional perspective view an alternative vent assembly 122. In particular, the example vent assembly 122 of FIG. 10 comprises a reservoir 1000 and a check valve 1002. The example check valve 1002 is shown as pinch valve, sometimes referred as a “duck billed” valve, though other check valve structures are possible. The example reservoir 1000 is fluidly coupled to the vent tube 120 (FIG. 1) by way of the check valve 1002. During filling of the ear canal with therapeutic fluid, displaced air and excess therapeutic fluid flow through the vent tube 120, and then through the check valve 1002, and then enter the reservoir 1000. The reservoir 1000 further defines an aperture or outlet 1004. Displaced air vents from the reservoir 1000 by way of the outlet 1004. A certain amount of excess therapeutic fluid may be held in the reservoir 1000 by surface tension, and the balance may flow out of the reservoir by way of the aperture 1004.

In accordance with example embodiments, the reservoir 1000 of FIG. 10 is implemented in a transparent material such that the clinician, injecting the therapeutic fluid into the ear canal (e.g., by way of the fill connector 118 and fill tube 116) can monitor the reservoir 1000. When therapeutic fluid arrives in the reservoir 1000, the clinician may assume the ear canal has been fully filled with the therapeutic fluid, and thus the filling may stop and the iontophoresis may proceed.

The example vent assembly 122 of FIG. 10 thus defines a coupler or connector 1006. The connector 1006 is designed and constructed to mechanically and fluidly couple to the vent tube 120 (FIG. 1) such that displaced air and excess therapeutic fluid may flow through check valve 1002 into the reservoir 1000. The example vent assembly 122 of FIG. 10 further comprises a cylinder or tube member 1008. The tube member 1008 couples to the connector 1006 on a first end, and as shown the connector 1006 and the tube member 1008 may sandwich or capture the check valve 1002 in place. The volume of the reservoir 1000 may take any suitable volume. In particular, the volume of the reservoir 1000 may be between and including 0.100 cubic centimeters cc and 0.300 cc. For all the example vent assemblies discussed to this point, one consideration is that venting of air and/or excess fluid can result in sound that is conveyed patient, and that sound may be perceived as loud given the direct mechanical/fluid connection to the tympanic membrane. In the case of vent assemblies, the opening or “crack” pressure for the check valve may be low to reduce the chance of mechanical noise being created and conveyed to the patient. In the examples of FIGS. 9 and 10, the check valve 902 in the form of the flap member 914 may have the lower crack pressure, and thus produce less sound. However, check valve 1002 may also be selected with a low crack pressure for the same reason.

Returning briefly to FIG. 1. The various embodiments of the earset assembly 100 discussed to this point have assumed the adhesion surface that holds the earset assembly 100 in place to be in a single location (e.g., pad 102). However, in yet still further embodiments the adhesion surface may be spread across several distinct locations. The specification now turns to alterative embodiments where the adhesion surface is spread across distinct locations.

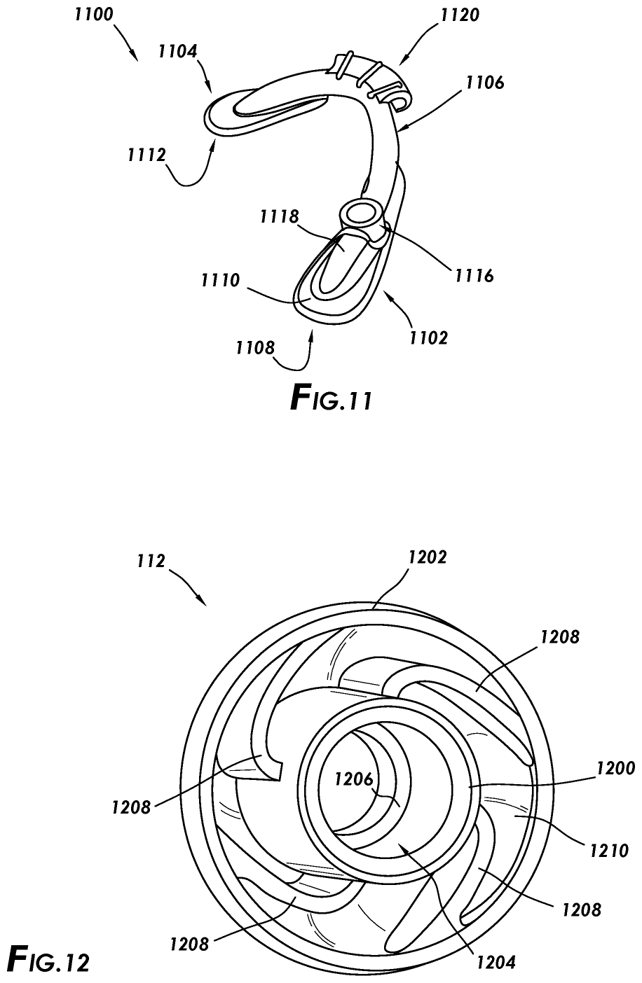

FIG. 11 shows a perspective view of a pad system 1100. In particular, the pad system 1100 defines a front pad 1102, a rear pad 1104, and a connecting arm 1106. The front pad 1102 defines a patient side 1108 (not visible) having an adhesive disposed thereon, in some cases a pressure sensitive adhesive. As before, the patient side 1108 is so named because, in use, the adhesive of the patient side 1108 adheres to the face of the patient. The front pad 1102 further defines a non-adhesive or clinician side 1110, opposite the patient side 1108. Again, the clinician side 1110 is so named to because, in use, the clinician side 1110 likely faces the clinician, particularly during installation. In example embodiments, the front pad 1102 defines a polygonal shape, and in particular a triangular shape with rounded corners, but other shapes are possible. The rear pad 1104 also defines a patient side 1112 (not visible) having an adhesive disposed thereon. The read pad 1104 further defines a non-adhesive or clinician side 1110, opposite the patient side 1112. In example embodiments, the rear pad 1104 defines a polygonal shape, and in particular a triangular shape with rounded corners, but other shapes are possible. The front pad 1102 and rear pad 1104 need not have the same overall shape.

The example pad system 1100 further defines a connection member or connecting arm 1106. The connecting arm 1106 mechanically couples the front pad 1102 and the read pad 1104, and holds the relative orientations of pads. That is, the front pad 1102 is coupled on a first end of the connecting arm 1106, and the rear pad 1104 is coupled on a second end of the connecting arm 1106, opposite the first end. When used as part of an earset assembly, the pad system 1100 is adhered to the face of the patient. The front pad 1102 may be placed on the skin of the patient's face near the tragus, such as in the side-burn region. The rear pad 1104 adheres to the skin behind the ear of the patient. The front pad 1102 is thus a first pad member, and the rear pad 1104 is thus a second pad member distinct from the first pad member. The connecting arm 1106 defines a radius of curvature designed and constructed to fit over the ear of a patient.

Still referring to FIG. 11, the example front pad 1102 further comprises a pad connector 1116 medially disposed on the front pad 1102, though other placements are possible. In particular, the front pad 1102 defines a reinforcing ridge 1118 that runs along the length of the front pad on the clinician side 1110. The reinforcing ridge 1118 not only reinforces the front pad 1102 to reduce flexing along the length, but also provides a location for the example pad connector 1116. The example pad connector 1116 may be of similar form and construction to pad connector 206 (FIG. 2), and those feature are not repeated again here so as not to unduly lengthen the discussion. Suffice it say that an arm 104 (FIG. 1) couples to the pad system 1100 in the fashion previously discussed for purposes of holding an earset 106 within the patient's ear canal.

FIG. 11 further shows that the pad system 1100 has an optional tube holder 1120 defined at the apex of the curvature of the connecting arm 1106. As the name implies, the tube holder 1120 is designed and constructed to hold the tube set 114 in place over and behind the patient's ear such that both filling with therapeutic fluid, and venting (if any) may take place at some distance from the ear of the patient. The tube holder 1120 may be omitted in some cases.

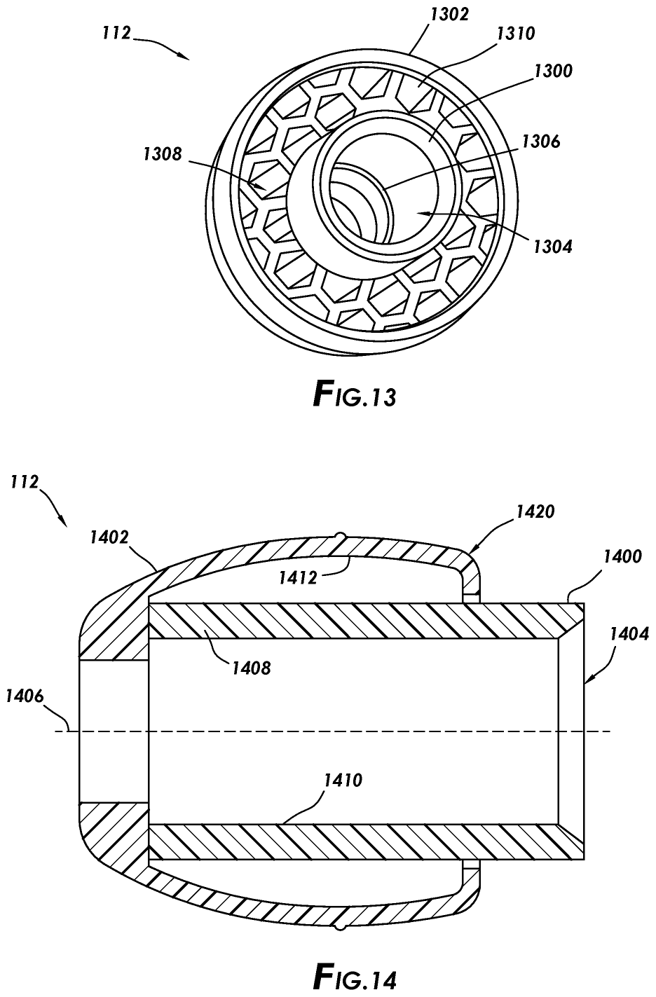

The specification now turns to a few further example ear plugs that include radial reinforcement to reduce or resist deformation in radial directions relative to or toward the longitudinal central axis of the ear plug. FIG. 12 show a rear perspective view of another example ear plug 112. In particular, visible in FIG. 12 is a tube portion 1200 and seal portion 1202. The internal lumen 1204 is shown, and a portion of an annular channel 1206 within the internal lumen 1204 is also visible. The example ear plug 112 includes the radial reinforcement, but in the example form of spokes 1208. In particular, the example ear plug 112 comprises spokes 1208 that extend from the outside diameter of the tube portion 1200 to the inside surface 1210 of the seal portion 1202. While four spokes 1208 are shown, any non-zero number of spokes 1208 may be implemented. The example spokes 1208 tend to resist movement of the inside surface 1210 of the seal portion 1202 toward the outside surface of the tube portion 1200. The example spokes are bent or curved, and thus tend pivot or hinge as radially inward force is applied to the seal portion 1202 (e.g., during insertion into an ear canal). For additional rigidity, the spokes 1208 may have less curvature, and in one example case the spokes may define straight walls that are parallel to outward projecting radials from a longitudinal central axis (not specifically shown) of the ear plug 112.

The example ear plug 112 of FIG. 12 may be constructed of single durometer rating elastomeric material. In other cases, the radial reinforcement may be implemented in a combination of the spokes in any suitable form and a dual durometer construction in which the tube portion 1200 and spokes 1208 are made of an elastomeric material having a higher durometer rating than the seal portion 1202. In other cases, the seal portion 1202 and spokes 1208 may have the same durometer rating, the durometer rating of the seal portion 1202 and spokes 1208 may be lower than the durometer rating of the tube portion 1200.

FIG. 13 shows a back perspective view of another example ear plug 112. In particular, visible in FIG. 13 is a tube portion 1300 and seal portion 1302. The internal lumen 1304 is shown, and a portion of an annular channel 1306 within the internal lumen 1304 is also visible. The example ear plug 112 includes the radial reinforcement, but in the example form of a honeycomb structure 1308. In particular, honeycomb structure 1308 comprises a series of stacked hexagon tubes that share walls between them, though any polygonal tube structure may be used. Each hexagonal tube defines a channel that is parallel to the longitudinal central axis of the internal lumen 1304. The honeycomb structure 1308 extends from the outside diameter of the tube portion 1300 to the inside surface 1310 of the seal portion 1302. The example honeycomb structure 1308 tends to resist movement of the inside surface 1310 of the seal portion 1302 toward the outside surface of the tube portion 1300. The example ear plug 112 of FIG. 13 may be constructed of single durometer rating elastomeric material. In other cases, the radial reinforcement may be implemented in a combination of the honeycomb structure and a dual durometer construction in which the tube portion 1300 and honeycomb structure 1308 are made of an elastomeric material having a higher durometer rating than the seal portion 1302. In other cases, the seal portion 1302 and honeycomb structure 1308 may have the same durometer rating, the durometer rating of the seal portion 1302 and honeycomb structure 1308 may be lower than the durometer rating of the tube portion 1300.

FIG. 14 shows a cross-sectional side view of another example ear plug 112. In particular, FIG. 14 shows the tube portion 1400 and the seal portion 1402. The tube portion 1400 defines an internal lumen 1404 and a longitudinal central axis 1406. The inside diameter of the example tube portion 1400 includes a counter bore that defines an annular shoulder 1408 near the distal end of the internal lumen 1404. Also defined on the inside diameter of the internal lumen 1404 is an annular channel 1410, and the annular channel 1410 is disposed proximally from the annular shoulder 1408.

The seal portion 1402 is coupled to the distal end of the tube portion 1400. The example seal portion 1402 extends distally from the tube portion 610, but also extends proximally over the tube portion 1400. The example seal portion 1402 defines a “tear drop” shape including an inside surface 1412 that faces the outside diameter or outside surface of the tube portion 1400. Stated otherwise, the seal portion 1402 defines a frusto-ellipsoid outside surface 1420, and a corresponding frusto-ellipsoid inside surface.

In the example ear plug 112 of FIG. 14, the radial reinforcement may be implemented by way of a dual-durometer construction. For example, the tube portion 1400 may be an elastomeric material having a durometer rating higher than a durometer rating the seal portion 1402, similar to the embodiments of FIG. 8 (including the ranges of durometer ratings for each). In additional to, or in place of, the dual-durometer design, the internal volume defined between the inside surface 1412 of the seal portion 1402 and the outside diameter of the tube portion 1400 may be filled with a compliant gel or suitable liquid that, by its viscosity, resists deformation of the seal portion 1402 toward the outside diameter of the tube portion 1400. In example cases where the ear plug 112 has a compliant gel in the internal volume, the tube portion 1400 and seal portion 1402 may be made of elastomeric material having about the same durometer rating.

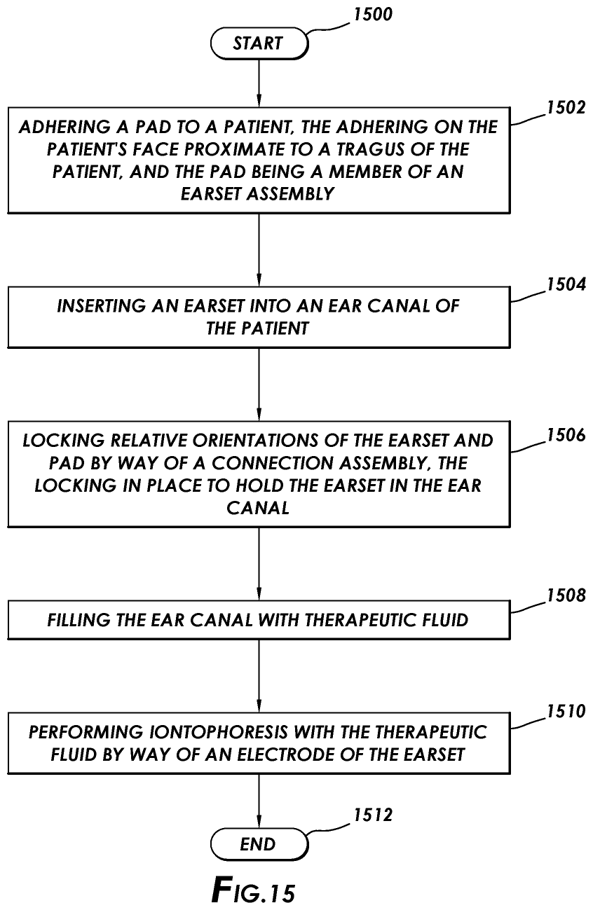

FIG. 15 shows a method in accordance with at least some embodiments. In particular, the method starts (block 1500) and comprises: adhering a pad to a patient, the adhering on the patient's face proximate to a tragus of the patient, and the pad being a member of an earset assembly (block 1502); inserting an earset into an ear canal of the patient (block 1504); locking relative orientations of the earset and pad by way of a connection assembly, the locking in place to hold the earset in the ear canal (block 1506); filling the ear canal with therapeutic fluid (block 1508); and performing iontophoresis with the therapeutic fluid by way of an electrode of the earset (block 1510). Thereafter the method ends (block 1512), likely to be restarted on the second ear of the patient.

The above discussion is meant to be illustrative of the principles and various embodiments of the present invention. Numerous variations and modifications will become apparent to those skilled in the art once the above disclosure is fully appreciated. For example, while FIG. 3 shows the socket 314 adjustable along the arm 104, and the arm being “fixed” to the pad 102, in yet still other cases the socket 314 may be rigidly coupled to the arm 104, and the arm 104 may have a variable connection to the pad 102 (e.g., using a similar locking knob arrangement). As another example, once iontophoresis is complete, the earset may be removed, but the pad and arm (rotated out of the way) may be remain in place while the procedure is performed on the other ear. It is intended that the following claims be interpreted to embrace all such variations and modifications.

1. An earset assembly comprising:
a pad defining an adhesive side having an adhesive, a non-adhesive side opposite the adhesive side;
an arm defining a proximal end and a distal end, the proximal end coupled to the non-adhesive side of the pad;
an earset comprising:
a rigid tube defining a proximal end, a distal end, and an internal volume;
a nozzle protruding from the internal volume and through the distal end of the rigid tube;
an electrode disposed within the internal volume; and
the proximal end of the rigid tube coupled to the arm.

2. The earset assembly of claim 1 further comprising a pad connector coupled between the proximal end of the arm and the non-adhesive side of the pad, the pad connector defining an axis of rotation for the arm, and the axis of rotation intersects the adhesive side of the pad.

3. The earset assembly of claim 1 further comprising a pad connector coupled between the proximal end of the arm and the non-adhesive side of the pad, the arm has a single degree of freedom relative to the pad.

4. The earset assembly of claim 1 further comprising an arm connector coupled between the arm and the earset, the arm connector provides the earset only three degrees of freedom relative to the arm.

5. The earset assembly of claim 1 further comprising an arm connector coupled between the arm and the earset, the arm connector forming a ball and a socket arrangement.

6. The earset assembly of claim 5 wherein the socket protrudes from a distal surface of the arm, and the ball protrudes from the proximal end of the earset.

7. (canceled)

8. The earset assembly of claim 5 further comprising sound damper disposed between the ball and the socket arrangement.

9. (canceled)

10. The earset assembly of claim 1 further comprising:
a slot defined through the arm;
an arm connector comprising a connector member abutting a patient side of the arm, and a knob member abutting the clinician side of the arm;
the arm connector comprising a first configuration in which the arm connector slides along the slot, and a second configuration in which the arm connector is rigidly coupled to the slot.

11.-12. (canceled)

13. The earset assembly of claim 1 wherein the earset further comprises an ear plug concentrically arranged over a distal end of the rigid tube, the ear plug comprising:
a tube portion defining an internal lumen and a longitudinal central axis, an inside diameter of the tube portion coupled to an outside diameter of the rigid tube of the earset;
a seal portion coupled to a distal end of the tube portion, and the seal portion extending proximally over the tube portion; and
the tube portion comprising a means for radial reinforcement to resist deformation in radial directions relative the longitudinal central axis.

14. The earset assembly of claim 13 wherein the means for radial reinforcement further comprises a plurality of spokes coupled between an inside surface of the seal portion and an outside surface of the tube portion.

15. The earset assembly of claim 13 wherein the means for radial reinforcement further comprises the tube portion having a durometer rating higher than a durometer rating of the seal portion.

16-18. (canceled)

19. The earset assembly of claim 1 wherein the earset further comprises:
a vent tube defining an internal vent path, a first end of the internal vent path fluidly coupled to the internal volume of the rigid tube; and
a check valve coupled to a second end of the internal vent path, the check valve configured to allow fluid to pass only out of the internal vent path on the second end.

20. The earset assembly of claim 19 wherein the earset further comprises a reservoir disposed between the internal vent path and the check valve.

21. The earset assembly of claim 20 wherein the reservoir is at least partially transparent.

22. The earset assembly of claim 19 wherein the earset further comprises a reservoir disposed downstream of the check valve.

23. The earset assembly of claim 22 wherein the reservoir is at least partially transparent.

24. The earset assembly of claim 19 wherein the earset further comprises a fill tube defining an internal flow path, a first end of the fill tube coupled to the internal volume of the rigid tube, and a second end coupled to a fill connector.

25. The earset assembly of claim 19 wherein the vent tube is at least five times longer than a length of the rigid tube of the earset.

26. The earset assembly of claim 1 wherein the pad further comprises a width and a length, and wherein the length is at least three times the width.

27. The earset assembly of claim 1 where the pad further comprises:
a first pad member, the first pad member having a first portion of the adhesive;
a second pad member distinct from the first pad member, the second pad member having a second portion of the adhesive;
a connection member defining a radius of curvature, the connection member having a first end coupled to the first pad member, and the connection member having a second end coupled to the second pad member, the second end opposite the first end.

28.-41. (canceled)
